(19) United States
(12) Patent Application Publication
(10) Pub. No.: US 2026/0002215 A1
(43) Pub. Date: Jan. 1, 2026

(54) ADAM32 GENE IN THE DIAGNOSIS AND TREATMENT OF LUNG ADENOCARCINOMA

(71) Applicant: Huazhong Agricultural University, Wuhan (CN)

(72) Inventors: Hongyu Zhang, Wuhan (CN); Yuan Quan, Wuhan (CN); Gang Wang, Wuhan (CN); Baixue Chen, Wuhan (CN); Xiaomin Zhang, Wuhan (CN); Min Gao, Wuhan (CN)

(21) Appl. No.: 18/755,893

(22) Filed: Jun. 27, 2024

(51) Int. Cl. C12Q 1/6886; C07K 16/28; C12N 15/113

(52) U.S. Cl. CPC C12Q 1/6886; C07K 16/2896; C12N 15/113; C07K 2317/73; C12N 2310/14; C12Q 2600/136; C12Q 2600/158

(57) ABSTRACT

The present application relates to a use of a disintegrin and metalloproteinase domain-containing protein 32 (ADAM32) gene in diagnosis and treatment of lung adenocarcinoma, and in particular to a product for diagnosis of lung adenocarcinoma that includes a detection reagent for the ADAM32 gene as a diagnostic marker for the lung adenocarcinoma. The present application determines a relationship between the ADAM32 gene and lung adenocarcinoma for the first time, that is, the inhibition on the expression of ADAM32 has an effect of delaying the progression of lung adenocarcinoma and prolonging a life span of a patient. The present application provides an effective novel way for the treatment of lung adenocarcinoma.

### TECHNICAL FIELD

The present application relates to the technical field of medicine, and specifically to a use of a disintegrin and metalloproteinase domain-containing protein 32 (ADAM32) gene in diagnosis and treatment of lung adenocarcinoma.

### REFERENCE TO SEQUENCE LISTING

The Sequence Listing XML file submitted via the USPTO Patent Center, with a file name of “Substitute_Sequence_Listing_SCH-24047-USPT.xml”, a creation date of Sep. 3, 2024, and a size of 13,476 bytes, is part of the specification and is incorporated in its entirety by reference herein.

### BACKGROUND

Lung tumor originates from the epithelium of a bronchial mucosa and can be classified into small cell lung cancer and non-small cell lung cancer. The non-small cell lung cancer includes squamous cell carcinomas, adenocarcinomas, and large cell carcinomas. The small cell lung cancer accounts for about 15% to 20% of bronchus-originated lung carcinomas. About 30% of patients diagnosed with small cell lung cancer are at the limited stage, and the remaining are at the extensive stage. When a tumor spreads to body parts above the clavicle, the tumor reaches an extensive stage. The non-small cell lung cancer has slower cell growth and division and later spread and metastasis than the small cell lung cancer. The non-small cell lung cancer accounts for about 80% of all lung carcinomas. About 75% of patients diagnosed with non-small cell lung cancer are at middle and advanced stages, and have a very low 5-year survival rate. Lung adenocarcinoma is one of the most common malignancies. Unlike squamous cell lung cancer, lung adenocarcinoma is likely to occur in women and non-smokers. Lung adenocarcinoma originates from an epithelium of a bronchial mucosa, and may also originate from the mucous gland of a large bronchus in a few cases. Lung adenocarcinoma has a lower incidence than squamous carcinomas and undifferentiated carcinomas. Lung adenocarcinoma occurs at the young age, and is common in women. Most adenocarcinomas originate from small bronchi and are peripheral lung cancers. Lung adenocarcinoma at the early stage usually does not have obvious clinical symptoms, and is often discovered during chest X-ray examination. Lung adenocarcinoma is manifested as a round or oval lump, which usually grows slowly, but sometimes undergoes hematogenous metastasis at the early stage. Lung adenocarcinoma undergoes lymphatic metastasis late. The traditional drugs targeting known targets can no longer meet the therapeutic needs, and there is an urgent need to explore novel therapeutic targets.

The disintegrin and metalloproteinase domain-containing protein 32 (ADAM32; UniProt ID: (Human) Q8TC27; Entrez Gene ID: (Human) 203102) gene is a protein-coding gene. ADAM32, which belongs to the ADAM protein family, is a transmembrane protein. An extracellular region of ADAM32 includes binding domains for metalloproteinase and integrin. ADAM32 is a catalyst-like protein. ADAM32 is highly expressed in testes and may play a role during sperm development and fertilization.

### SUMMARY

In view of the current clinical demand for drugs targeting novel targets to prevent and treat lung adenocarcinoma, an objective of the present application is to provide an ADAM32 gene as a novel target for diagnosis and treatment of lung adenocarcinoma. The ADAM32 gene can be used to predict the prognosis of a lung adenocarcinoma patient and can be used as a therapeutic target for lung adenocarcinoma.

A first aspect of the present application provides a product for diagnosis of lung adenocarcinoma, including a detection reagent for an ADAM32 gene as a diagnostic marker for the lung adenocarcinoma. The present application determines a relationship between the ADAM32 gene and lung adenocarcinoma for the first time, that is, the inhibition on the expression of ADAM32 has an effect of delaying the progression of lung adenocarcinoma and prolonging a life span of a patient. The present application provides an effective novel way for the treatment of lung adenocarcinoma.

In some embodiments, the detection reagent is a reagent for determining an expression level of the ADAM32 gene in a biological sample. Further, the biological sample is a human lung cell.

In some embodiments, the reagent for determining the expression level of the ADAM32 gene in the biological sample includes a reagent for detecting a messenger RNA (mRNA) of the ADAM32 gene or a fragment thereof, a reagent for detecting a complementary DNA (cDNA) of the ADAM32 gene or a fragment thereof, and/or a reagent for detecting a protein encoded by the ADAM32 gene or a variant thereof.

In some embodiments, the reagent for determining the expression level of the ADAM32 gene in the biological sample includes primers for the ADAM32 gene and/or an ADAM32 antibody. Further, the primers for the ADAM32 gene are an upstream primer set forth in SEQ ID NO: 9 (5′-ACCCAAACACAAAGCAGTAG-3′, SEQ ID NO: 9) and a downstream primer set forth in SEQ ID NO: 10 (5′-GACCACGGAAGGAAAGACAG-3′, SEQ ID NO: 10).

In some embodiments, detection reagents required for detecting the expression level of the ADAM32 gene through real-time fluorescent quantitative polymerase chain reaction (PCR) include: All-in-one First Strand cDNA Synthesis Kit (gDNA Purge), KAPA SYBR FAST qPCR Master Mix (2×), and primers for ADAM32 (an upstream primer sequence: ACCCAAACACAAAGCAGTAG, SEQ ID NO: 9; and a downstream primer sequence: GACCACGGAAGGAAAGACAG, SEQ ID NO: 10).

In some embodiments, detection reagents required for detecting an expression level of an ADAM32 protein through immunohistochemistry include: an ADAM32 antibody (THERMO FISHER SCIENTIFIC, Item No.: PA5-83709) and a Dako REAL EnVision Detection System (AGILENT, Item No.: K5007).

In some embodiments, the product is a chip, a kit, or a test paper.

A second aspect of the present application provides a drug for treating lung adenocarcinoma, including a reagent for inhibiting expression of an ADAM32 gene. The present application determines a relationship between the ADAM32 gene and lung adenocarcinoma for the first time, that is, the inhibition on the expression of ADAM32 has an effect of delaying the progression of lung adenocarcinoma and prolonging a life span of a patient. The present application provides an effective novel way for the treatment of lung adenocarcinoma.

In some embodiments, the reagent for inhibiting the expression of the marker ADAM32 includes an siRNA specifically targeting the ADAM32 gene, an antisense oligonucleotide specifically targeting the ADAM32 gene, a ribozyme specifically targeting the ADAM32 gene, and/or an inhibitory antibody for the ADAM32 gene.

In some embodiments, the inhibitory antibody for the ADAM32 gene is a monoclonal antibody, a polyclonal antibody, or a polyspecific antibody. In some embodiments, the inhibitory antibody for the ADAM32 gene is the polyclonal antibody, and an antigen targeted by the polyclonal antibody is CHO-derived recombinant human ADAM32 Ser17-Thr476. In some embodiments, the inhibitory antibody for the ADAM32 gene is an ADAM32 antibody (Item No.: PA5-83709) of Thermo Fisher Scientific.

In some embodiments, nucleotide sequences for the siRNA specifically targeting the ADAM32 gene are SEQ ID NOS: 1 and 2, SEQ ID NOS: 3 and 4, or SEQ ID NOS: 5 and 6. Further, the nucleotide sequences for the siRNA specifically targeting the ADAM32 gene are SEQ ID NOS: 3 and 4.

A third aspect of the present application provides a drug screening system, including: (1) a detection module configured to detect an inhibitory effect of a drug on expression of an ADAM32 gene, and (2) an analysis module configured to output therapeutic effect data of the drug for treating lung adenocarcinoma according to detection data output by the detection module.

A fourth aspect of the present application provides a lung adenocarcinoma diagnosis system, including: (1) a detection device configured to detect an expression level of an ADAM32 gene in a biological sample of a subject, and (2) an analysis device configured to output a diagnosis result of lung adenocarcinoma of the subject according to data of the expression level of the ADAM32 gene output by the detection device. Further, the biological sample is a human lung cell.

Compared with the prior art, the present application has the following advantages and effects: The present application determines a relationship between ADAM32 and lung adenocarcinoma for the first time, that is, the inhibition on ADAM32 has an effect of preventing, alleviating, or treating lung adenocarcinoma. Based on the close relationship between ADAM32 and lung adenocarcinoma, ADAM32 can be used as a novel target for drugs to treat lung adenocarcinoma, such that a novel treatment method or drug suitable for the treatment of lung adenocarcinoma can be screened, which can effectively solve the problem that it is difficult to screen drugs in the prior art. An inhibitor for ADAM32 can be used to prepare a drug for treating lung adenocarcinoma.

### BRIEF DESCRIPTION OF THE DRAWINGS

FIG. 1 shows the survival analysis results of the ADAM32 gene of the present application in the TCGA-HNSC dataset;

FIG. 2 shows the comparison of immunohistochemical scores of the ADAM32 gene of the present application in lung adenocarcinoma and a paracancerous tissue;

FIG. 3 shows the verification of interfering effects of small interfering fragments of the ADAM32 gene of the present application in NCI-H1299 cells (fluorescent quantitative PCR);

FIG. 4 shows the experimental results of an impact of interfering the ADAM32 gene of the present application on the proliferation of NCI-H1299 cells;

FIG. 5 shows the experimental results of an impact of interfering the ADAM32 gene of the present application on the proliferation of NCI-H1395 cells;

FIG. 6 shows the experimental results of an impact of interfering the ADAM32 gene of the present application on the scratch assay of NCI-H1299 cells;

FIG. 7 shows the experimental results of an impact of interfering the ADAM32 gene of the present application on the scratch assay of NCI-H1395 cells;

FIG. 8 shows the experimental results of an impact of interfering the ADAM32 gene of the present application on the apoptosis of NCI-H1395 cells;

FIG. 9 shows the experimental results of an impact of interfering the ADAM32 gene of the present application on the cycle of NCI-H1395 cells;

FIG. 10 shows the experimental results of an impact of interfering the ADAM32 gene of the present application on the invasion of NCI-H1395 cells;

FIG. 11 shows the experimental results of the inhibition of the incubation of the ADAM32 gene of the present application with an antibody at different concentrations on the proliferation of NCI-H1395 cells (namely, an IC₅₀ determination experiment);

FIG. 12 shows the experimental results of an impact of the incubation of the ADAM32 gene of the present application with an antibody at 10 μg/mL in NCI-H1395 cells on the migration of cells;

FIG. 13 shows the experimental results of an impact of the incubation of the ADAM32 gene of the present application with an antibody at 10 μg/mL in NCI-H1395 cells on the invasion of cells;

FIG. 14 shows the experimental results of reactive oxygen species (ROS) by preliminary mechanism analysis-flow cytometry;

FIG. 15 shows the experimental results of mitochondrial membrane potential by preliminary mechanism analysis-flow cytometry;

FIG. 16 shows the CCK8 test results of an impact of an expression level of the ADAM32 gene of the present application on the growth of organoids;

FIG. 17 shows the ATP activity test results of the sensitivity of ADAM32 of the present application to an ADAM32 antibody; and

FIG. 18 shows the ATP activity test results for screening an ADAM32 antibody drug with lung cancer organoids in the present application.

### DETAILED DESCRIPTION

To well explain the objective, technical solutions, and advantages of the present application, the present application will be further explained below with reference to the accompanying drawings and specific examples. It should be understood by those skilled in the art that the specific examples described herein are merely intended to explain the present application, rather than to limit the present application.

### Ohnolog Genes

With the development of evolutionary biomedicine, the accumulated evolution knowledge has been successfully used to analyze the pathogenesis of various diseases and identify the pathogenic genes. Since the evolution of genes is closely related to the occurrence and progression of various diseases (especially cancer), a variety of studies have shown that successful drug targets often have common evolutionary characteristics. Whole-genome duplication is often considered as an important evolutionary event in vertebrates. The ancient human genome underwent two times of whole-genome duplication during the early vertebrate period. To commemorate the outstanding contributions of Ohno to related studies, the genes associated with the two whole-genome duplication events are called “Ohnologs”. The Ohnolog genes play an important role in the development and transcriptional regulation of organisms. Previous studies have shown that the Ohnolog genes are closely related to the occurrence and progression of human phenotypes or diseases (including cancer) due to high dose sensitivity (a change in a gene copy number will lead to a phenotypic change). Because a qualified drug target gene needs to be closely associated with a phenotype of a disease and needs to be sensitive to a drug stimulation, the dose sensitivity characteristics of the Ohnolog genes make the Ohnolog genes promising drug targets. In addition, it is well known that cancer cells can escape cell division and programmed death control through “rapid” evolution, which leads to the rapid spread of cancer. A large number of studies have also shown that cancer-associated genes have unique evolutionary origin stages. Therefore, the tracing of an origin of a cancer gene to inhibit or slow down an evolutionary process of the cancer gene is an idea to effectively reduce the adaptability of cancer cells to fight against the progression of cancer. Studies have shown that cancer driver genes are significantly enriched in cellular organisms, eukaryotes, opisthokonta, and eumetazoa at origin stages. The research by the inventor team of the present application has shown that ADAM32 not only is an Ohnolog gene, but also originates from a cancer-associated origin stage of eumetazoa. However, there are no reports on the association of ADAM32 with lung adenocarcinoma and the use of ADAM32 in screening drugs for prevention and treatment of lung adenocarcinoma.

### Example 1 Survival analysis results of an ADAM32 gene in a TCGA-HNSC dataset

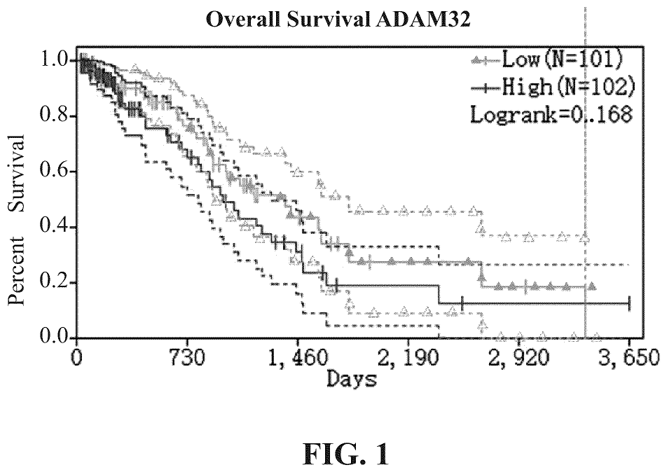

Specifically, the following process was provided:
(1) Analysis of evolutionary characteristics of the ADAM32 gene: Results showed that ADAM32 was an Ohnolog gene, ADAM32 originated from an stage of evolutionary origin of eumetazoa, and ADAM32 met the evolutionary characteristics of cancer genes.
(2) By querying the DriverDBv3 database of cancer driver genes, it could be known that ADAM32 was a cancer driver gene.
(3) By querying the TissGDB database of cancer tissue-specific expressed genes, it could be known that ADAM32 was a cancer tissue-specific expressed gene, and was highly expressed specifically in a cancer tissue.
(4) By querying the UniProt database, it could be known that the subcellular localization of a protein encoded by ADAM32 was on a cell membrane.
(5) Multiomics data of lung adenocarcinoma patients (TCGA-HNSC) was downloaded from the Cancer Genome Atlas (TCGA) database (https://www.cancer.gov/tcga). The data included gene expression data and clinical data of the patients. The clinical data included a survival status and a survival time, and the gene expression data included a gene expression level FPKM determined by RNA sequencing (RNA-seq).
(6) TCGA-HNSC data in the step (5) was pre-processed to obtain a gene expression matrix of human genes in the lung adenocarcinoma patients, and a Cox proportional hazards regression model was established between an expression level FPKM of the ADAM32 gene and a prognostic survival time of a patient. Results showed that an expression level FPKM of the ADAM32 gene was significantly correlated with a survival time of a lung adenocarcinoma patient (P-value=4.77E-03), with a hazard ratio of 6.6679 (FIG. 1). These results indicate that the high expression of ADAM32 is a risk factor threatening a survival time of a lung adenocarcinoma patient, that is, the inhibition on ADAM32 can alleviate the recurrence of lung adenocarcinoma and prolong a life span of a patient.

### Example 2

In this example, a protein expression level of ADAM32 in a lung adenocarcinoma tissue was analyzed by an immunohistochemical staining experiment.

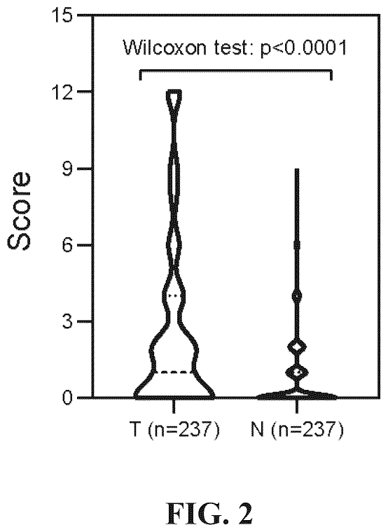

A total of 474 paraffin-embedded pathological samples were collected from lung adenocarcinoma patients clinically, including 237 pairs of cancerous and paracancerous samples. 5 μm-thick paraffin sections were prepared. The paraffin sections each were subjected to deparaffinization and hydration, incubated with 3% H₂O₂ for 10 min to block an activity of endogenous peroxidase, then subjected to high-pressure and microwave antigen retrieval with the antigen retrieval Tris-EDTA solution (pH 9.0), cooled naturally, and then blocked with 10% goat serum for 20 min. After the blocking was completed, the blocking solution was removed with an absorbent paper, then an ADAM32 antibody (purchased from Thermo Fisher Scientific, Item No.: PA5-83709) diluted at 1:200 was added dropwise to each section to fully cover a tissue, and each section was incubated overnight at 4° C. The next day, each section was soaked in a 0.05% Tween-20-containing PBS solution 3 times for 3 min to 5 min each time, then Dako REAL En Vision/HRP (purchased from Agilent, Item No.: K5007ENV) was added dropwise to each section to fully cover a tissue, and each section was incubated at 37° C. for 20 min. Each section was soaked in a 0.05% Tween-20-containing PBS solution 4 times for 3 min to 5 min each time. A Dako DAB solution was added to each section to allow a chromogenic reaction for 5 min to 10 min. After the chromogenic reaction was completed, each section was thoroughly cleaned, incubated with a hematoxylin staining solution for 3 min, washed with tap water, differentiated with 1% hydrochloric acid-ethanol for 15 s, and then soaked in a saturated disodium phosphate solution for 1 min to allow blue returning. Each counterstained section was blow-dried (or air-dried) with a blow dryer, then mounted with a neutral gum, observed and photographed under a microscope, and scored for a staining intensity. Results showed that an expression level and a positive rate of ADAM32 in a lung adenocarcinoma tissue were significantly higher than those in a paracancerous tissue (FIG. 2).

### Example 3

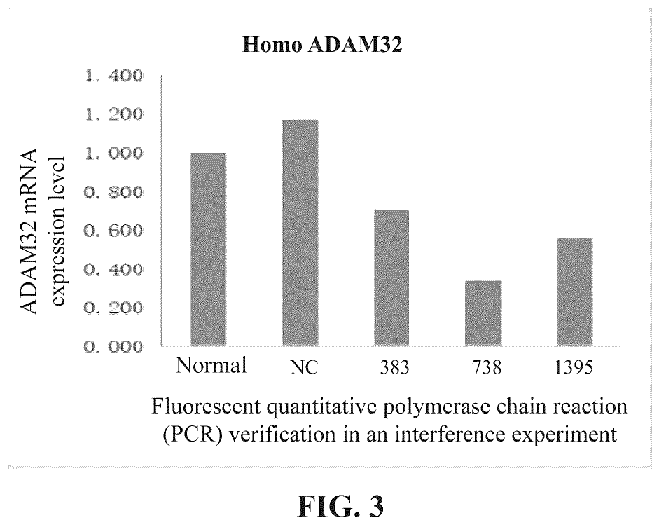

For the ADAM32 gene, three siRNAs and one NC (negative control) RNA were designed and synthesized, and corresponding sequences were shown in Table 1. A human lung cancer cell line was transfected with each sequence for 24 h. A total of 5 groups were set, including a blank cell group. The optimal sequence ADAM32-Homo-738 was obtained through PCR screening (FIG. 3). NCI-H1299 cells (purchased from Procell) and NCI-H1395 cells (purchased from Procell) were selected and recovered with an NCI-H1299 cell culture medium and an NCI-H1395 cell culture medium, respectively. When a cell density reached 80%, cells were passaged. Then cells with a prominent growth state in a logarithmic growth phase were collected for a cell transfection experiment, where parallel controls were set. NCI-H1299 cell groups: (1) a normal control for NCI-H1299 cells, (2) NCI-H1299 cells+NC siRNA (NC group), and (3) NCI-H1299 cells+ADAM32-Homo-738 (ADAM32 siRNA). NCI-H1395 cell groups: (1) a normal control for NCI-H1395 cells, (2) NCI-H1395 cells+NC siRNA (NC group), and (3) NCI-H1395 cells+ADAM32-Homo-738 (ADAM32 siRNA). After the 24 h of transfection, transfected cells were subjected to CCK8 assay and scratch assay. The NCI-H1299 cells (human non-small cell lung cancer cells) were purchased from Procell. The NCI-H1299 cells were derived from a patient with lymph node metastasis who received initial radiotherapy. The NCI-H1299 cells partially lacked the p53 protein homogeneously, and did not express the p53 protein. The NCI-H1299 cells could synthesize neuromedin B (NMB) at 0.1 pmol/mg, and did not synthesize gastrin-releasing peptide (GRP). The NCI-H1395 cells (human lung adenocarcinoma cells) were purchased from Procell. The NCI-H1395 line was established in April 1986, and a corresponding tissue provider smoked 15 packs per year. NCI-BL1395 [BL1395] cells were a lymphoblast strain derived from the same patient.

TABLE 1

| | Sequence | No. |
|---|---|---|
| ADAM32-Homo-383 | Sense strand: 5-GCUCUGGACUAAGAGGAAUTT-3 | SEQ ID NO: 1 |
| | Antisense strand: 5-AUUCCUCUUAGUCCAGAGCTT-3 | SEQ ID NO: 2 |
| ADAM32-Homo-738 | Sense strand: 5-GCUGUCAUCAUUGGAGUUATT-3 | SEQ ID NO: 3 |
| | Antisense strand: 5-UAACUCCAAUGAUGACAGCTT-3 | SEQ ID NO: 4 |
| ADAM32-Homo-1395 | Sense strand: 5-GCCGAAAGCACAUCCUGAATT-3 | SEQ ID NO: 5 |
| | Antisense strand: 5-UUCAGGAUGUGCUUUCGGCTT-3 | SEQ ID NO: 6 |
| NC sequence | Sense strand: 5-UUCUCCGAACGUGUCACGUTT-3 | SEQ ID NO: 7 |
| | Antisense strand: 5-ACGUGACACGUUCGGAGAATT-3 | SEQ ID NO: 8 |

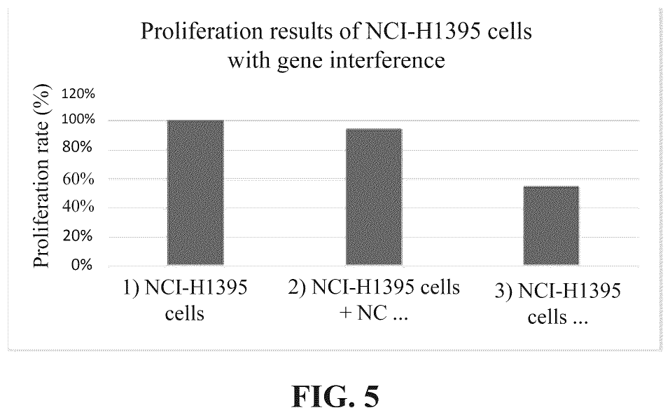

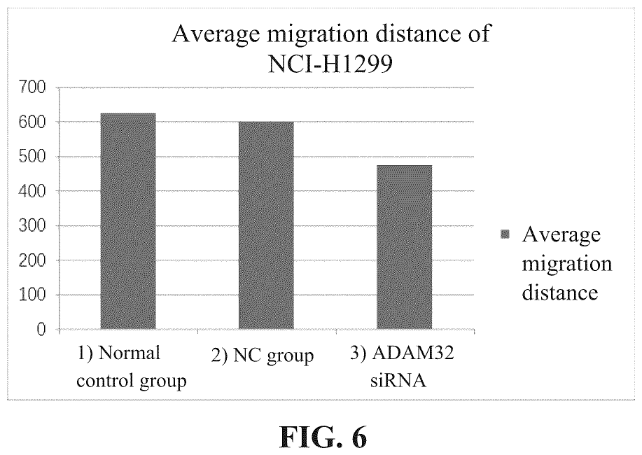

CCK8 assay: After cell culture was completed, 10 μL of CCK8 was added to each well and cells were further cultivated at 37° C. for 1 h to 2 h. The absorbance OD₄₅₀ of each well was determined by a microplate reader. Experimental results were sorted and then shown in FIG. 5 and FIG. 6.

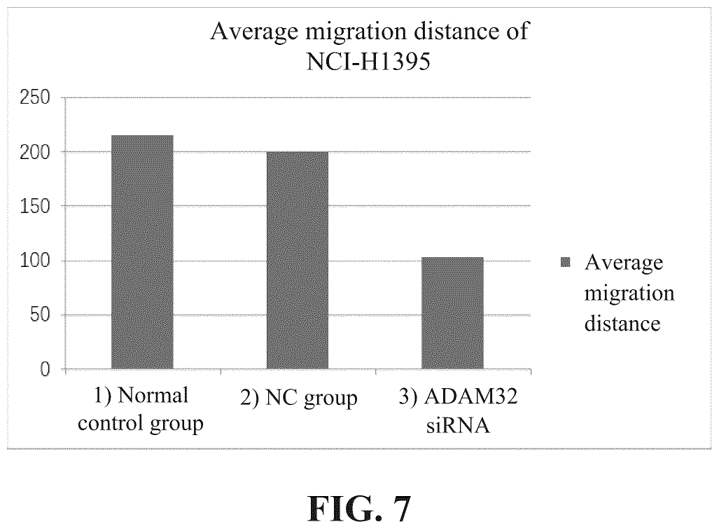

Scratch assay: Two groups of experiments were conducted in parallel for the above two cell lines. A marker pen was used to draw horizontal lines on a back of a 6-well plate along a ruler evenly at an interval of about 0.5 cm to 1 cm, where each well was crossed by three horizontal lines. Cells were digested with 0.25% trypsin, then inoculated at 2×10⁶ cells/well into the 6-well plate, and cultivated for 24 h at 37° C., 5% CO₂, and a saturated humidity. When a cell density reached about 90% and a bottom of the 6-well plate was almost fully covered by cells, scratches perpendicular to the horizontal lines on the back were created with a pipette tip along the ruler, where a same pipette tip was used to create scratches crossing different wells. The cells were washed 3 times with PBS to remove the scratched cells, then a serum-free medium was added to the plate, and cells were photographed (0 h), then cultivated in a 37° C. and 5% CO₂ incubator for 24 h, and then photographed. Results of the scratch assay were sorted and then shown in FIG. 6 and FIG. 7.

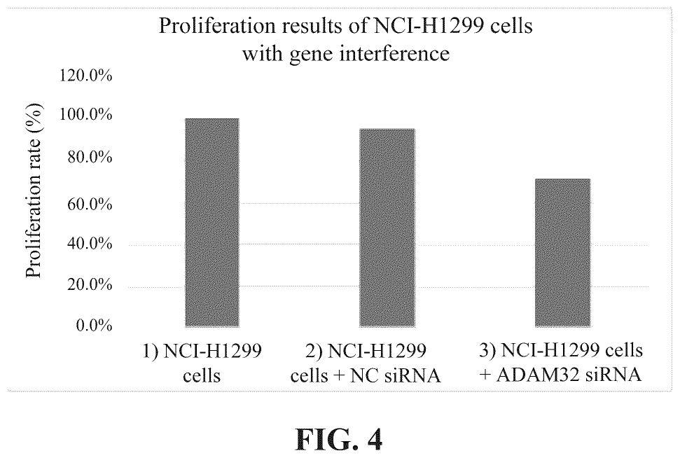

The results showed that proliferation abilities (as shown in FIG. 4 and FIG. 5) and migration abilities (as shown in FIG. 6 and FIG. 7) of NCI-H1299 cells and NCI-H1395 cells in which the ADAM32 gene was interfered with/inhibited were reduced compared with a proliferation ability and a migration ability of control cells in which the ADAM32 gene was not interfered with.

A cryopreservation tube with NCI-H1395 cells was taken out from liquid nitrogen, quickly placed in a 37° C. water bath, and gently shaken until the cryopreservation solution was thawed. The cells were transferred to a centrifuge tube with 5 mL of a medium and centrifuged for 5 min at 1,000 rpm and room temperature to obtain a supernatant and a cell pellet, and the supernatant was discarded. The cell pellet was suspended in a 10% fetal bovine serum (FBS)-containing complete medium, inoculated into a petri dish, gently pipetted up and down for thorough mixing, and cultivated at 37° C., 5% CO₂, and a saturated humidity. Cell passage and expanded culture: When a cell density reached 80%, cells were passaged as follows: A medium was removed, and the cells were washed once with PBS. 1 mL to 2 mL of 0.25% trypsin was added to digest the cells for 1 min to 2 min. When it was observed under a microscope that the cells were separated from each other and rounded, the trypsin was quickly removed, the complete medium was added, and cells were pipetted up and down to obtain a single-cell suspension. The cells were passaged at a ratio of 1:3, and subjected to expanded culture at 37° C., 5% CO₂, and a saturated humidity. Cell treatment: NCI-H1395 cells with a prominent growth state in a logarithmic growth phase were collected and prepared with a 1640 medium into a single-cell suspension, evenly inoculated into a 6-well plate at 2× 10⁵ cells/well, and cultivated overnight at 37° C., 5% CO₂, and a saturated humidity. 2 h before transfection, the 1640 medium was replaced with a serum-free 1640 medium. The transfection was conducted according to the following experimental groups:
1) NCI-H1395 cell control group;
2) ADAM32 siRNA interference control group; and
3) ADAM32 siRNA interference group.

Transfection: Each transfected sample was prepared as follows: 10 μL of a small fragment (concentration: 20 μM) was diluted with 100 μL of serum-free Opti-MEM, gently pipetted up and down for thorough mixing, and allowed to stand at room temperature for 5 min to obtain a small fragment dilution. Lipofectamine™ 2000 was gently mixed thoroughly before use, and then 5 μL of the Lipofectamine™ 2000 was taken, diluted with 100 μL of Opti-MEM, and allowed to stand for 5 min at room temperature to obtain a Lipofectamine™ 2000 dilution. The Lipofectamine™ 2000 dilution and the small fragment dilution (total volume: 200 μL) were gently mixed thoroughly and allowed to stand for 20 min at room temperature to obtain a mixed solution. 200 μL of the mixed solution was added to each culture well, and a corresponding cell culture plate was gently shaken back and forth to make the mixed solution thoroughly mixed with a culture in the cell culture plate. Cells were cultivated in a 37° C. and 5% CO₂ incubator for 6 h, and then the transfection solution was replaced with the normal medium. The cells were further cultivated in a 37° C. and 5% CO₂ incubator for 24 h and then subjected to subsequent tests.

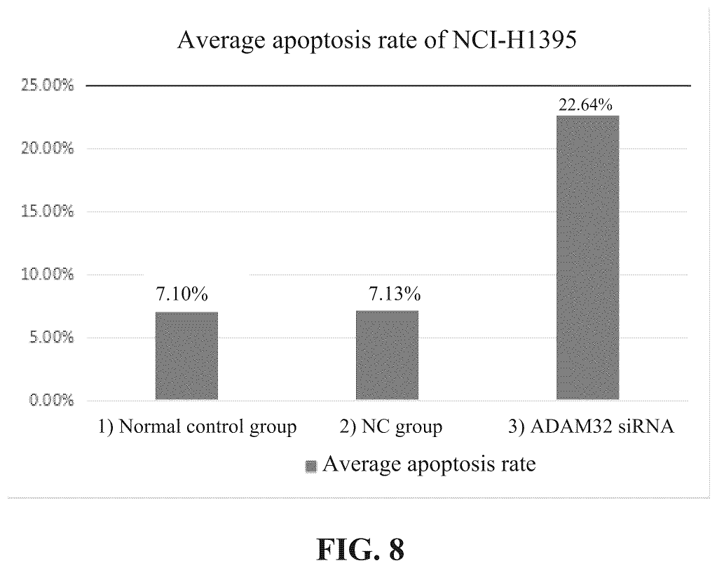

Apoptosis test: Cells were digested with EDTA-free 0.25% trypsin, then the digestion was terminated to obtain a digestion system, and the digestion system was centrifuged at 1,500 rpm for 5 min to obtain a supernatant and a cell pellet. The supernatant was discarded, and the cell pellet was suspended with PBS. Cells were rinsed 2 times with PBS, where the centrifugation was conducted at 1500 rpm for 5 min. The cells were tested with an AnnexinV-APC/7-AAD apoptosis test kit as follows: 50 μL of Binding Buffer and 5 μL of a 7-AAD staining solution were thoroughly mixed to obtain a mixed solution. The mixed solution was thoroughly mixed with the collected cells, and a reaction was conducted at room temperature in the dark for 5 min to 15 min. Then 450 μL of Binding Buffer was added, and thorough mixing was conducted. 5 μL of Annexin V-APC was added, thorough mixing was conducted, and a reaction was conducted at room temperature in the dark for 5 min to 15 min. A product was tested by a flow cytometer. Experimental results were shown in FIG. 8.

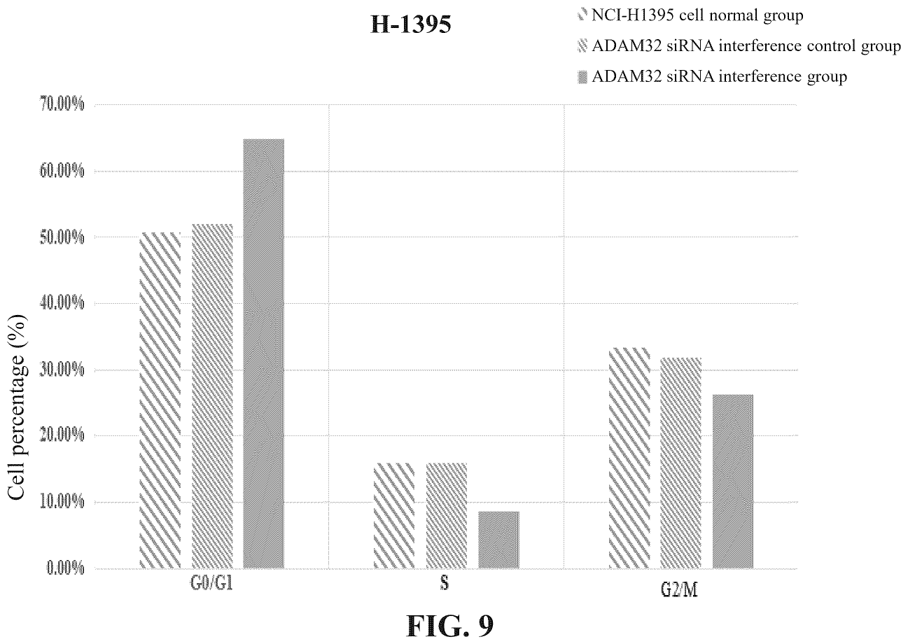

Cell cycle test: Treated cells were digested with EDTA-free 0.25% trypsin, then the digestion was terminated to obtain a digestion system, and the digestion system was centrifuged at 1,000 rpm for 5 min to obtain a first supernatant and a first cell pellet. The first supernatant was discarded, and the first cell pellet was suspended and rinsed 2 times with PBS, where the centrifugation was conducted at 1,000 rpm for 5 min to finally obtain a second supernatant and a second cell pellet. The second supernatant was discarded. The second cell pellet was suspended with 100 μL of PBS, 700 μL of pre-cooled 80% ethanol was slowly added with a final ethanol concentration of 70%, and fixation was conducted at 4° C. for 4 h or more to obtain a fixation system. The fixation system was centrifuged at 1,000 rpm for 5 min to obtain a third cell pellet, and the third cell pellet was rinsed 2 times with pre-cooled PBS to obtain a cell suspension. 100 μL of RNase (50 μg/mL) was added to the cell suspension to obtain a first mixture, and the first mixture was incubated in a 37° C. water bath for 30 min to obtain a second mixture. 400 μL of PI (50 μg/mL) was added to the second mixture, and staining was conducted at 4° C. in the dark for 30 min. A product was tested by a flow cytometer. Experimental results were shown in Table 2 and FIG. 9.

TABLE 2

| | Proportions of cells | | |
|---|---|---|---|
| | in different phases, % | | |
| | G1 | S | G2 |
| Group | phase | phase | phase |
| NCI-H1395 cell control group | 50.71% | 16.05% | 33.24% |
| ADAM32 siRNA interference control group | 52.13% | 15.91% | 31.96% |
| ADAM32 siRNA interference group | 64.92% | 8.68% | 26.4% |

NCI-H1395 cells were selected and recovered with an NCI-H1395 cell culture medium, and when a cell density reached 80%, the cells were passaged. Then NCI-H1395 cells with a prominent growth state in a logarithmic growth phase were collected and prepared with a 1640 medium into a single-cell suspension, evenly inoculated into a 6-well plate at 2× 10⁵ cells/well, and cultivated overnight at 37° C., 5% CO₂, and a saturated humidity. 2 h before transfection, the 1640 medium was replaced with a serum-free 1640 medium. The transfection was conducted according to the following experimental groups:
1) normal cell control group;
2) ADAM32 siRNA interference control group;
3) ADAM32 siRNA interference group; and
4) ADAM32 antibody group (concentration: 10 μg/mL, and treatment time: 24 h).

Transfection: Each transfected sample was prepared as follows: 10 μL of a small fragment (concentration: 20 μM) was diluted with 100 μL of serum-free Opti-MEM, gently pipetted up and down for thorough mixing, and allowed to stand at room temperature for 5 min to obtain a small fragment dilution. Lipofectamine™ 2000 was gently mixed thoroughly before use, and then 5 μL of the Lipofectamine™ 2000 was taken, diluted with 100 μL of Opti-MEM, and allowed to stand for 5 min at room temperature to obtain a Lipofectamine™ 2000 dilution. The Lipofectamine™ 2000 dilution and the small fragment dilution (total volume: 200 μL) were gently mixed thoroughly and allowed to stand for 20 min at room temperature to obtain a mixed solution. 200 μL of the mixed solution was added to each culture well, and a corresponding cell culture plate was gently shaken back and forth to make the mixed solution thoroughly mixed with a culture in the cell culture plate. Cells were cultivated in a 37° C. and 5% CO₂ incubator for 6 h, and then the transfection solution was replaced with the normal medium. The cells were further cultivated in a 37° C. and 5% CO₂ incubator for 24 h and then subjected to subsequent tests.

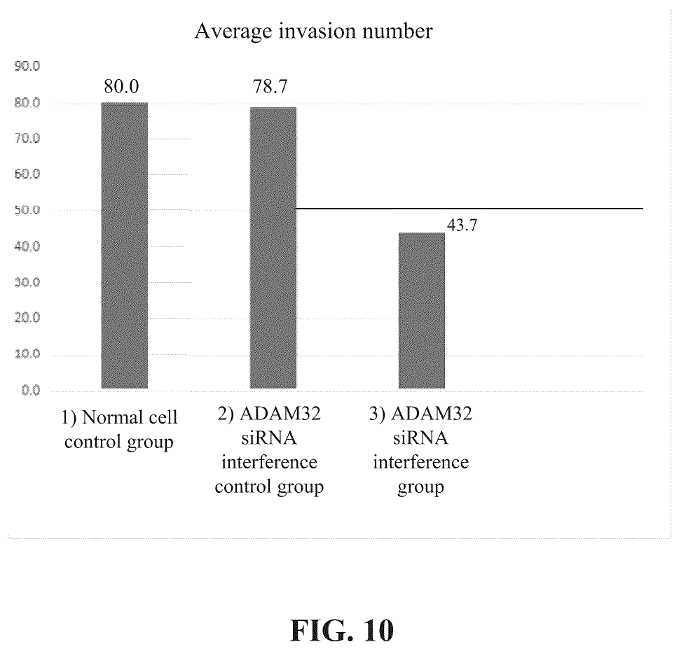

The treated NCI-H1395 cells were washed with 3 mL of PBS and digested with 0.25% trypsin to obtain a digestion system, and the digestion system was centrifuged at 1,000 rpm for 5 min to obtain a supernatant and a cell pellet. The supernatant was discarded, and the cell pellet was rinsed with PBS twice to remove residual serum. Cells were suspended with a serum-free 1640 medium, counted on a counting board, and diluted with the serum-free 1640 medium to a cell concentration of 3× 10⁵ cells/mL for later use. Matrigel was thawed at 4° C. one day in advance, and transwell chambers, 24-well plates, and pipette tips were pre-cooled overnight at −20° C. The Matrigel was diluted with a serum-free medium to a final concentration of 1 mg/mL on ice. 800 μL of a 10% FBS-containing 1640 medium (including penicillin-streptomycin) pre-cooled at 4° C. was added to a 24-well plate, then a transwell chamber was placed in the 24-well plate, 100 μL of the Matrigel with a final concentration of 1 mg/mL was vertically added to a center of a bottom of an upper chamber of the transwell chamber, and the plate was incubated at 37° C. for 4 h to 5 h until the Matrigel was solidified. Then, 200 μL of a cell suspension for each group was added to the upper chamber of the transwell chamber, and the plate was incubated in a 37° C. and 5% CO₂ incubator for 24 h. The transwell chamber was taken out and carefully washed once with PBS. Cells were fixed with a 70% ice ethanol solution for 1 h, stained with a 0.5% crystal violet staining solution, placed at room temperature for 20 min, and washed once with PBS. The unmigrated cells at a side of the upper chamber were wiped off with a clean cotton ball. Cells were observed and photographed under a microscope. Experimental results of an impact of the interference on the invasion were shown in FIG. 10.

NCI-H1395 cells were selected and recovered with an NCI-H1395 cell culture medium, and when a cell density reached 80%, the cells were passaged. Then NCI-H1395 cells with a prominent growth state in a logarithmic growth phase were collected, inoculated into a 96-well plate at 5×10³ cells/well, and cultivated overnight at 37° C., and a blank group was set (100 μL of sterile PBS was added to a well around a cell well). Cells were treated according to the following groups: NCI-H1395 normal group; NCI-H1395 cells+1.5625 μg/mL ADAM32 antibody; NCI-H1395 cells+3.125 μg/mL ADAM32 antibody; NCI-H1395 cells+6.25 μg/mL ADAM32 antibody; NCI-H1395 cells+12.5 μg/mL ADAM32 antibody; NCI-H1395 cells+25 μg/mL ADAM32 antibody; and NCI-H1395 cells+50 g/mL ADAM32 antibody. The cells were treated for 24 h. CCK8 assay of cells: After the cells were cultivated for a desired time, 10 μL of cck8 was added to each well and cells were further cultivated at 37° C. for 1 h to 4 h. The absorbance OD₄₅₀ of each well was determined by a microplate reader.

Scratch assay: A marker pen was used to draw horizontal lines on a back of a 6-well plate along a ruler evenly at an interval of about 0.5 cm to 1 cm, where each well was crossed by three horizontal lines. Cells were digested with 0.25% trypsin, then inoculated at 2× 10⁶ cells/well into the 6-well plate, and cultivated for 24 h at 37° C., 5% CO₂, and a saturated humidity. When a cell density reached about 90% and a bottom of the 6-well plate was almost fully covered by cells, scratches perpendicular to the horizontal lines on the back were created with a pipette tip along the ruler, where a same pipette tip was used to create scratches crossing different wells. The cells were washed 3 times with PBS to remove the scratched cells, then a serum-free medium was added to the plate, and cells were photographed (0 h) and treated as follows: 1) normal control for NCI-H1395 cells; and 2) NCI-H1395 cells+ADAM32 antibody (10 μg/mL). The treated cells were cultivated in a 37° C. and 5% CO₂ incubator for 24 h, and then photographed. The ADAM32 antibody (PA5-83709) was purchased from Thermo Fisher Scientific and was an antigen-immunized polyclonal antibody, where the antigen was CHO-derived recombinant human ADAM32 Ser17-Thr476.

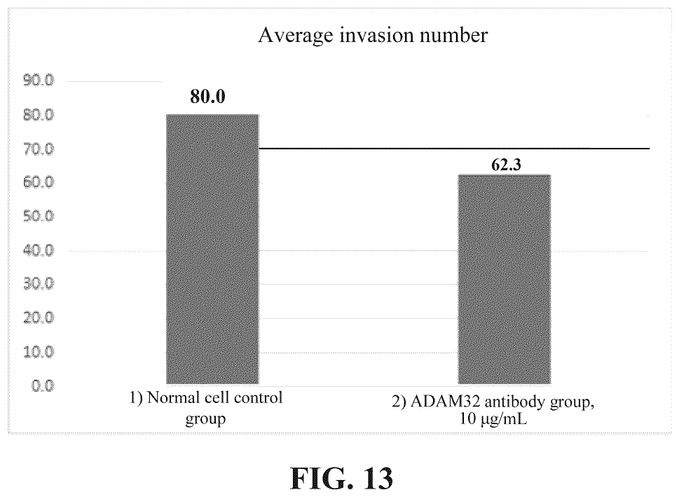

A cell invasion experiment was conducted by the same steps as above. An impact of antibody incubation on the invasion was shown in FIG. 13.

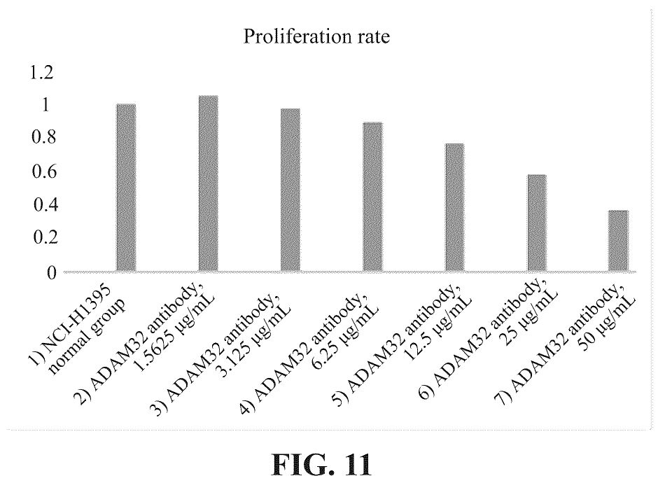

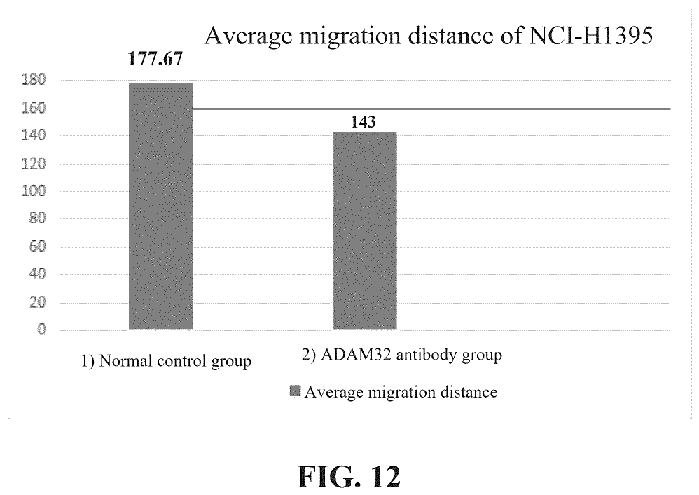

Experimental results showed that a proliferation rate of lung cancer cells after being incubated with the ADAM32 antibody was reduced. IC₅₀ test results were shown in FIG. 11. The antibody concentration of 10 μg/mL was selected for incubation. A proliferation rate of NCI-H1395 cells after being incubated with the ADAM32 antibody was reduced by about 20% (FIG. 11). A migration rate of NCI-H1395 cells after being incubated with the ADAM32 antibody was decreased by about 20% (FIG. 12). An invasion ability of NCI-H1395 cells after being incubated with the ADAM32 antibody was decreased by about 20% (FIG. 13). The specific ADAM32 antibody could inhibit the proliferation, migration, and invasion of lung adenocarcinoma to allow an effect of treating lung adenocarcinoma.

Then, a target mechanism was preliminarily analyzed by the inventors. Through RNAseq transcriptome sequencing before and after the ADAM32 gene was interfered with and KEGG pathway enrichment analysis, it was found that, after the ADAM32 gene was interfered with, an oxidative phosphorylation pathway in cells was abnormal, and genes for pathways such as mitochondrial oxidative phosphorylase, respiratory electron transport, and chemiosmotic coupling to synthesize ATP were up-regulated. Whether the death of cancer cells is mediated by a mitochondrial apoptosis pathway was preliminarily analyzed. The mitochondrial apoptosis pathway refers to the opening of mitochondrial permeability transition pores mediated by reactive oxygen species (ROS), which makes a mitochondrial membrane system undergo a structural change and breakup and fragmentation and may cause the cell death in a severe case. In order to preliminarily prove the above conjecture, ROS and a mitochondrial membrane potential were detected by flow cytometry to preliminarily explain an apoptosis mechanism, and a cellular ROS detection experiment and a cellular JC-1 experiment were conducted.

A cryopreservation tube with NCI-H1395 cells was taken out from liquid nitrogen, quickly placed in a 37° C. water bath, and gently shaken until the cryopreservation solution was thawed. The cells were transferred to a centrifuge tube with 5 mL of a medium and centrifuged for 5 min at 1,000 rpm and room temperature to obtain a supernatant and a cell pellet, and the supernatant was discarded. The cell pellet was suspended in a 10% FBS-containing complete medium, inoculated into a petri dish, gently pipetted up and down for thorough mixing, and cultivated at 37° C., 5% CO₂, and a saturated humidity.

### Cell Passage and Expanded Culture:

When a cell density reached 80%, cells were passaged as follows: The medium was removed, and the cells were washed once with PBS. 1 mL to 2 mL of 0.25% trypsin was added to digest the cells for 1 min to 2 min. When it was observed under a microscope that the cells were separated from each other and rounded, the trypsin was quickly removed, the complete medium was added, and cells were pipetted up and down to obtain a single-cell suspension. The cells were passaged at a ratio of 1:3, and subjected to expanded culture at 37° C., 5% CO₂, and a saturated humidity.

### Cell Treatment:

NCI-H1395 cells with a prominent growth state in a logarithmic growth phase were collected and prepared with a 1640 medium into a single-cell suspension, evenly inoculated into a 6-well plate at 2× 10⁵ cells/well, and cultivated overnight at 37° C., 5% CO₂, and a saturated humidity. 2 h before transfection, the 1640 medium was replaced with a serum-free 1640 medium. The transfection was conducted according to the following experimental groups:
1) normal cell control group;
2) ADAM32 siRNA interference control group;
3) ADAM32 siRNA interference group; and
4) ADAM32 antibody group (concentration: 10 μg/mL, and treatment time: 24 h). Transfection:

Each transfected sample was prepared as follows: 10 μL of a small fragment (concentration: 20 μM) was diluted with 100 μL of serum-free Opti-MEM, gently pipetted up and down for thorough mixing, and allowed to stand at room temperature for 5 min to obtain a small fragment dilution.

Lipofectamine™ 2000 was gently mixed thoroughly before use, and then 5 μL of the Lipofectamine™ 2000 was taken, diluted with 100 μL of Opti-MEM, and allowed to stand for 5 min at room temperature to obtain a Lipofectamine™ 2000 dilution. The Lipofectamine™ 2000 dilution and the small fragment dilution (total volume: 200 μL) were gently mixed thoroughly and allowed to stand for 20 min at room temperature to obtain a mixed solution. 200 μL of the mixed solution was added to each culture well, and a corresponding cell culture plate was gently shaken back and forth to make the mixed solution thoroughly mixed with a culture in the cell culture plate. Cells were cultivated in a 37° C. and 5% CO₂ incubator for 6 h, and then the transfection solution was replaced with the normal medium. The cells were further cultivated in a 37° C. and 5% CO₂ incubator for 24 h and then subjected to subsequent tests.

### Cellular ROS Detection:

Cells were digested with EDTA-free 0.25% trypsin, then the digestion was terminated to obtain a digestion system, and the digestion system was centrifuged at 1,500 rpm for 5 min to obtain a supernatant and a cell pellet. The supernatant was discarded. The cell pellet was suspended and rinsed with PBS twice, where the centrifugation was conducted at 1,500 rpm for 5 min. Cells were tested with a DCFH-DA cellular ROS test kit as follows: PBS was removed and 1 mL of diluted DCFH was added. The cells were incubated in a 37° C. incubator for 20 min during which thorough mixing was conducted once every 3 min. The cells were washed three times with a serum-free medium. The cells were suspended with 500 μL of PBS and then tested by a flow cytometer.

### Cellular JC-1 Detection:

Cells were digested with EDTA-free 0.25% trypsin, then the digestion was terminated to obtain a digestion system, and the digestion system was centrifuged at 1,200 rpm for 5 min to obtain a first supernatant and a first cell pellet. The first supernatant was discarded. The first cell pellet was suspended in 0.5 mL of a medium including serum and phenol red to obtain a cell suspension. 0.5 mL of a JC-1 staining working solution was added to the cell suspension to obtain a first mixed solution, and the first mixed solution was inverted up and down several times for thorough mixing and incubated in an incubator at 37° C. for 20 min. During an incubation period, an appropriate amount of a JC-1 staining buffer (1×) was prepared by adding 4 mL of distilled water per 1 mL of a JC-1 staining buffer (5×) and placed in an ice bath. After the 37° C. incubation was completed to obtain a second mixed solution, the second mixed solution was centrifuged at 1,200 rpm and 4° C. for 3 min to obtain a second supernatant and a second cell pellet. The second supernatant was discarded. The second cell pellet was washed twice with the JC-1 staining buffer (1×), then suspended with 1 mL of the JC-1 staining buffer (1×), and centrifuged for 3 min at 1,200 rpm and 4° C. to obtain a third supernatant and a third cell pellet. The third supernatant was discarded. The third cell pellet was suspended with 1 mL of the JC-1 staining buffer (1×) and centrifuged for 3 min at 1,200 rpm and 4° C. to obtain a fourth supernatant and a fourth cell pellet. The fourth supernatant was discarded. The fourth cell pellet was suspended with 500 μL of the JC-1 staining buffer (1×) and tested by a flow cytometer.

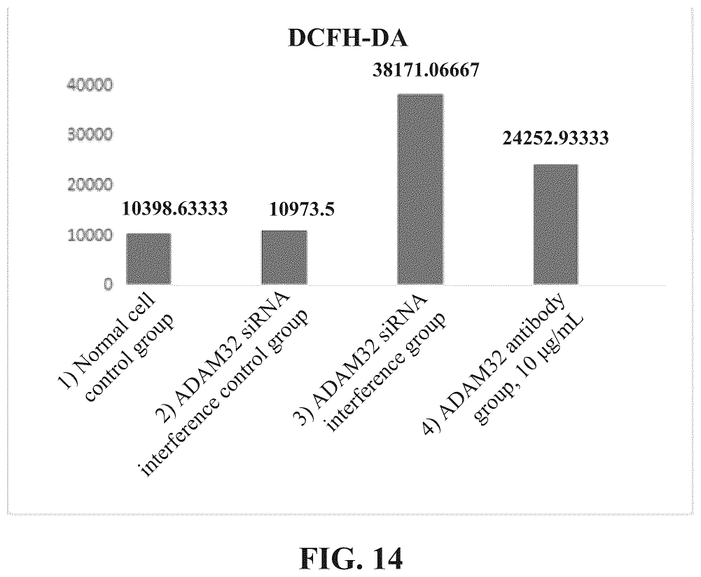

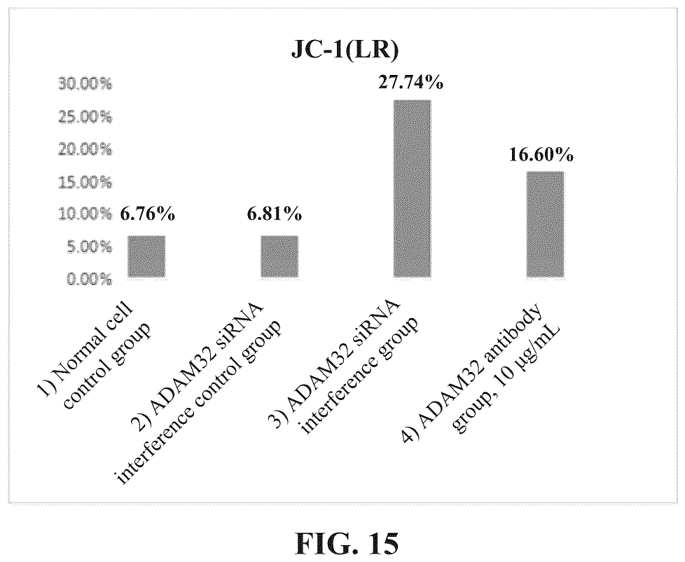

Experimental results showed that the interference of a drug/gene on ADAM32 affected a mitochondrial function in lung adenocarcinoma cells, and led to an increase of ROS in mitochondria (FIG. 14) and an abnormal membrane potential (FIG. 15).

### Example 4

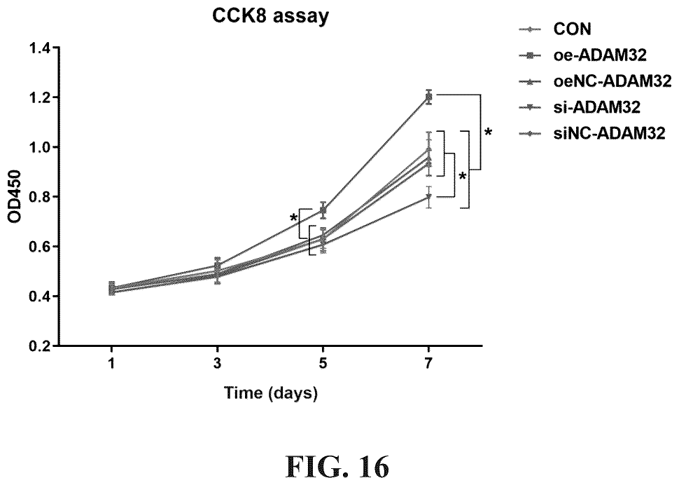

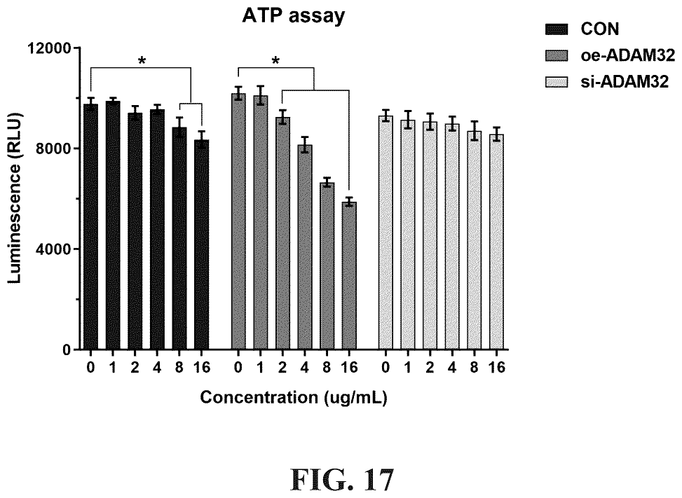

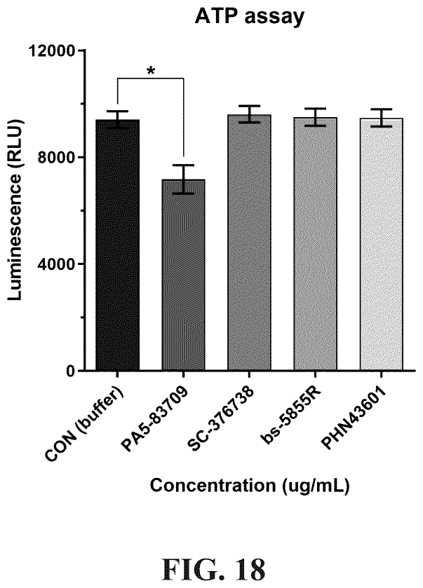

Tumor organoids are three-dimensional cell clusters produced from a patient-derived tumor tissue under special culture conditions, and retain the phenotypes such as histopathological, genetic, and molecular biological characteristics of an original tumor. Due to high clinical relevance, tumor organoid models can be used for screening of drug targets, which can facilitate the discovery of novel effective drugs and the prediction of drug responses. In this example, an experimental protocol for verifying an impact of the expression of the ADAM32 gene on the growth of lung adenocarcinoma using lung adenocarcinoma organoids, an experimental protocol for analyzing the sensitivity of an expression level of ADAM32 to an ADAM32 antibody, and an experimental protocol for screening ADAM32 antibody drugs using lung cancer organoids.
1. Primary culture of lung adenocarcinoma organoids: Lung adenocarcinoma organoids were cultivated using an Organotial human lung cancer organoid culture kit (purchased from ABSIN, Item No.: abs9443-1kit). A culture protocol was as follows:
   (1) Lung adenocarcinoma samples were collected clinically, placed in a pre-cooled tissue preservation solution, and transported to a clean laboratory for tissue treatment and cell isolation.
   (2) A tissue was placed in a petri dish, washed with a pre-cooled primary culture buffer 3 times to remove impurities, and cut with ophthalmic scissors into tissue pieces with a volume of about 1 mm³.
   (3) The tissue pieces were digested for 10 min to 20 min with a digestion solution for the human primary lung cancer tissue at 37° C. under shaking, and then the pre-cooled buffer for primary culture was added in a volume 3 times a volume of the tissue pieces to terminate the digestion to obtain a first cell suspension.
   (4) The first cell suspension was filtered through a screen mesh with a pore size of 100 μm to obtain a filtrate, and the filtrate was centrifuged at 300 g for 5 min to obtain a first supernatant and a first cell pellet. The first supernatant was discarded. The first cell pellet was suspended with 1 mL of a buffer for primary culture and then centrifuged to obtain a second supernatant and a second cell pellet, and the second cell pellet was collected.
   (5) A centrifuge tube with the second cell pellet was transferred into an ice box, and Matrigel (purchased from ABSIN, Item No.: abs9495) was added in a volume 25 times a volume of the second cell pellet to the centrifuge tube to resuspend cells to obtain a second cell suspension.
   (6) The second cell suspension was inoculated into a 24-well cell culture plate at 25 μL/well.
   (7) The cell culture plate was incubated in a 37° C. incubator for 20 min to 35 min until the Matrigel was solidified. A medium for the human lung cancer organoid was taken out from a refrigerator and equilibrated to room temperature.
   (8) The medium for the human lung cancer organoid was added at 0.5 mL/well to the 24-well cell culture plate.
   (9) The cell culture plate was incubated in a 37° C. incubator for 12 d to 14 d during which the original medium was replaced with a pre-warmed fresh medium for the human lung cancer organoid every other day.
2. Subculture of lung adenocarcinoma organoids: After the 12 d to 14 d of culture, the subculture could be conducted according to the following protocol:
   (1) The original medium was removed, 0.5 mL of a pre-cooled organoid subculture buffer was added to each well, and the plate was placed for 2 min. The matrigel was broken through gentle pipetting up and down using a pipette to obtain a suspension of lung adenocarcinoma organoids, and then the suspension of lung adenocarcinoma organoids was transferred to a 15 mL centrifuge tube.
   (2) 10 mL of a pre-cooled organoid subculture buffer was added to the centrifuge tube, the centrifuge tube was placed in a refrigerator at −20° C. for 3 min and then centrifuged at 4° C. and 400 g for 5 min to obtain a first supernatant and an organoid pellet, and the first supernatant was discarded.
   (3) The organoid pellet was digested with an organoid primary digestion solution for 2 min to 3 min, and a pre-cooled organoid subculture buffer was added in a volume 3 times a volume of the organoid pellet to terminate the digestion to obtain a digestion system. The digestion system was centrifuged at 4° C. and 400 g for 5 min to obtain a second supernatant and a first cell pellet, and the second supernatant was discarded.
   (4) The first cell pellet was suspended with 1 mL of a medium for the human lung cancer organoid, transferred to a 1.5 mL sterile centrifuge tube, and centrifuged at 4° C. and 300 g for 5 min to obtain a third supernatant and a second cell pellet. The third supernatant was discarded, and the second cell pellet was collected.
   (5) Organoid inoculation: The same steps as the steps (5) to (8) of primary culture of lung adenocarcinoma organoids were adopted.
   (6) The cell culture plate was incubated in a 37° C. incubator for 7 d to 9 d during which the original medium was replaced with a pre-warmed fresh medium for the human lung cancer organoid every other day. Then the passage, the cryopreservation, or the subsequent experiment could be conducted once again.
3. Lentiviral transfection of lung adenocarcinoma organoids: Lung cancer organoids with high or low expression of the ADAM32 gene were constructed through lentiviral transfection. Lentiviruses adopted were constructed by Genechem. The full sequence of ADAM32 was transfected with an over-expressed lentivirus, and the ADAM32-Homo-738 sequence in Example 2 was transfected with an interfering lentivirus. Implementation steps were as follows:
   (1) Organoid digestion: The original medium was removed, and a pre-cooled organoid subculture buffer was added. The matrigel was broken through gentle pipetting up and down using a pipette to obtain an organoid suspension, and then the organoid suspension was transferred to a 15 mL centrifuge tube.
   (2) 10 mL of a pre-cooled organoid subculture buffer was added to the centrifuge tube, the centrifuge tube was placed in a refrigerator at −20° C. for 3 min and then centrifuged at 4° C. and 400 g for 5 min to obtain a first supernatant and an organoid pellet, and the first supernatant was discarded.
   (3) The organoid pellet was digested with an organoid primary digestion solution for 3 min to 10 min such that about 80% of cells were digested into single cells, and a pre-cooled organoid subculture buffer was added in a volume 3 times a volume of the organoid pellet to terminate the digestion to obtain a digestion system. The digestion system was centrifuged at 4° C. and 400 g for 5 min to obtain a second supernatant and a first cell pellet, and the second supernatant was discarded.
   (4) The first cell pellet was suspended with 1 mL of a medium for the human lung cancer organoid, transferred to a 1.5 mL sterile centrifuge tube, and centrifuged at 4° C. and 300 g for 5 min to obtain a third supernatant and a second cell pellet. The third supernatant was discarded, and the second cell pellet was collected.
   (5) Cell inoculation: The second cell pellet was suspended with a medium for the human lung cancer organoid, counted, inoculated into a 24-well plate at 2× 10⁵ cells/well, and cultivated overnight in a 37° C. incubator.
   (6) Lentiviral transfection: The original medium was removed, and a fresh HiTransG P-containing medium was added. The over-expressed lentivirus, interfering lentivirus, and corresponding control lentivirus suspensions each were added according to MOI=30, and cells were further cultivated overnight in a 37° C. incubator.
   (7) After 20 h to 24 h of transfection, the original medium was removed, a pre-warmed medium for the human lung cancer organoid was added, and cells were further cultivated in a 37° C. incubator.
   (8) After 2 d of culture, the original medium was removed, lentivirus-transfected organoids were digested with an organoid primary digestion solution for 2 min to 5 min, and a pre-cooled organoid subculture buffer was added in a volume 3 times a volume of the lentivirus-transfected organoids to terminate the digestion to obtain a digestion system. The digestion system was centrifuged at 4° C. and 400 g for 5 min to obtain a fourth supernatant and a third cell pellet, and the fourth supernatant was discarded.
   (9) The third cell pellet was suspended with 1 mL of a medium for the human lung cancer organoid, transferred to a 1.5 mL sterile centrifuge tube, and centrifuged at 4° C. and 300 g for 5 min to obtain a fifth supernatant and a fourth cell pellet. The fifth supernatant was discarded, and the fourth cell pellet was collected.
   (10) Organoid inoculation: The same steps as the steps (5) to (8) of primary culture of lung adenocarcinoma organoids were adopted.
   (11) The cell culture plate was incubated in a 37° C. incubator for 24 h, and then the original medium was replaced with a fresh 1.5 μg/mL puromycin-containing medium.
   (12) The cell culture plate was further incubated in a 37° C. incubator for 5 d to 7 d during which the original medium was replaced with a fresh 1.5 μg/mL puromycin-containing medium every other day. Then the passage, the cryopreservation, or the subsequent experiment could be conducted once again. It should be noted that a medium used during the subsequent culture or experiment needs to include a half of the amount of puromycin (0.75 μg/mL) to prevent gene deletion.
4. CCK8 assay: An impact of an expression level of ADAM32 on the growth of organoids was analyzed through CCK8 assay.
   (1) Experimental groups: A normal group (CON) in which no lentivirus was transfected, an ADAM32 over-expression group (oe-ADAM32), an ADAM32 interference group (si-ADAM32), and a corresponding control lentivirus group (oeNC-ADAM32, siNC-ADAM32) were designed.
   (2) Organoid digestion: The same steps as the steps (1) to (4) of lentiviral transfection of lung adenocarcinoma organoids were adopted.
   (3) Cell inoculation: An organoid pellet obtained after digestion was suspended with a medium for the human lung cancer organoid, counted, inoculated at 5,000 cells/100 L/well into a 96-well plate, and cultivated overnight in a 37° C. incubator. 16 replicate wells were set for each group to allow detection at 4 time points of day 1, day 3, day 5, and day 7. It should be noted that a circle of blank wells were left around an experimental well with cells, and 200 μL of sterile PBS was added to each well to prevent a liquid in the experimental well from evaporating.
   (4) CCK8 test: After cells were cultivated for a predetermined time, 10 μL of a CCK8 reagent was added to each of 4 experimental wells for each group at each time point, the plate was incubated at 37° C. for 3 h, and the absorbance at 450 nM (OD₄₅₀) in each well was determined by a microplate reader. For other experimental wells that had not yet been used for detection, 10 μL of a fresh medium was added to each well every two days.
   (5) Experimental results: The absorbance values of the ADAM32 over-expression group on day 5 and day 7 were significantly higher than those of the other groups. The absorbance value of the ADAM32 interference group on day 7 was significantly lower than those of the other groups. There was no significant difference between the normal group and the control group at each time point. It can be seen that the high expression of ADAM32 could significantly promote the proliferation of lung cancer organoids, and the inhibition of expression of ADAM32 could significantly reduce a proliferation rate of lung cancer organoids. The experimental results were shown in FIG. 16.
5. ATP activity assay: The sensitivity of an expression level of ADAM32 to an ADAM32 antibody was analyzed through organoid ATP activity assay.
   (1) Experimental groups: A normal group (CON) in which no lentivirus was transfected, an ADAM32 over-expression group (oe-ADAM32), and an ADAM32 interference group (si-ADAM32) were designed.
   (2) Organoid digestion: The same steps as the steps (1) to (4) of lentiviral transfection of lung adenocarcinoma organoids were adopted.
   (3) A centrifuge tube with a cell pellet was transferred into an ice box, and Matrigel (purchased from ABSIN, Item No.: abs9495) was added in a volume 25 times a volume of the cell pellet to the centrifuge tube to resuspend cells to obtain a cell suspension.
   (4) The cell suspension was inoculated into a 384-well cell culture plate at 4 μL/well.
   (5) The cell culture plate was incubated in a 37° C. incubator for 20 min to 35 min until the Matrigel was solidified. A medium for the human lung cancer organoid was taken out from a refrigerator and equilibrated to room temperature.
   (6) Media for the human lung cancer organoids including 0 μg/mL, 1 μg/mL, 2 μg/mL, 4 μg/mL, 8 μg/mL, and 16 μg/mL of the ADAM32 antibody (purchased from Thermo Fisher Scientific, Item No.: PA5-83709) respectively were added to the 384-well cell culture plate at 40 μL/well. 3 replicate wells were set for each concentration. It should be noted that two circles of blank wells were left around an experimental well with cells, and 40 μL of sterile PBS was added to each well to prevent a liquid in the experimental well from evaporating.
   (7) Reagent preparation: An organoid ATP viability assay kit was purchased from Absin with Item No.: abs50059. 1 tube of a lyophilized powder of the organoid ATP fluorescent dye was added to 1 tube of an organoid ATP dye buffer of 10 mL, and the two were thoroughly mixed to prepare a detection reagent for the organoid ATP luminescence method. The reagent could be prepared on the day of ATP activity assay, or could be prepared and stored at −80° C. in the dark with an expiration date of one month.
   (8) ATP activity assay: After cells were cultivated for 7 d, the original medium in each experimental well was removed, 25 μL of the detection reagent for the organoid ATP luminescence method was added to each experimental well, and the plate was shaken on a microplate shaker to allow incubation for 10 min at room temperature. The chemiluminescence detection was conducted with a TECAN SPARK multi-mode microplate reader.
   (9) Experimental results: Chemiluminescence values of the ADAM32 over-expression group at ADAM32 antibody concentrations of 2 μg/mL, 4 μg/mL, 8 g/mL, and 16 μg/mL were significantly lower than those of the 0 μg/mL and 1 μg/mL groups, and there was a negative correlation between chemiluminescence values and antibody concentrations. The ADAM32 antibody at different concentrations did not have a significant impact on a chemiluminescence value of the ADAM32 interference group. Chemiluminescence values of the normal group at ADAM32 antibody concentrations of 8 μg/mL and 16 μg/mL were significantly lower than that of the 0 μg/mL group. It can be seen that the ADAM32 antibody could significantly inhibit the growth of organoids, and the higher the expression of ADAM32, the more sensitive to the ADAM32 antibody. Experimental results were shown in FIG. 17.
6. The screening method of ADAM32 antibody drugs: An experimental protocol for screening ADAM32 antibody drugs using lung cancer organoids was provided.
   (1) ADAM32-overexpressed organoid digestion and inoculation: The same steps as the steps (2) to (5) of ATP activity assay were adopted.
   (2) Antibody preparation: 4 ADAM32 antibodies were purchased from Thermo Fisher Scientific (Item No.: PA5-83709), Santa (Item No.: SC-376738), Bioss (Item No.: bs-5855R), and BiolabReagents (Item No.: PHN43601) respectively, and used to prepare media for the human lung cancer organoids including different ADAM32 antibodies at a concentration of 5 μg/mL. In addition, a medium for the human lung cancer organoid including an equal volume of an antibody buffer was prepared as a negative control.
   (3) A medium for the human lung cancer organoid including the ADAM32 antibody or the antibody buffer was added to the 384-well cell culture plate at 40 μL/well. 3 replicate wells were set for each group. It should be noted that two circles of blank wells were left around an experimental well with cells, and 40 μL of sterile PBS was added to each well to prevent a liquid in the experimental well from evaporating.
   (4) ATP activity detection: The same steps as the steps (7) and (8) of ATP activity assay were adopted.
   (5) Experimental results: A chemiluminescence value of ADAM32-overexpressed organoids intervened with the PA5-83709 antibody at 5 μg/mL was significantly lower than a chemiluminescence value of the antibody buffer control group. The intervention of other antibodies had no significant impact on a chemiluminescence value of ADAM32-overexpressed organoids. Experimental results were shown in FIG. 18. This experiment verified the feasibility of screening ADAM32 antibody drugs with lung cancer organoids.

Finally, it should be noted that the above examples are provided merely to describe the technical solutions of the present application, rather than to limit the protection scope of the present application. Although the present application is described in detail with reference to preferred examples, a person of ordinary skill in the art should understand that modifications or equivalent replacements may be made to the technical solutions of the present application without departing from the spirit and scope of the technical solutions of the present application.

1. A product for diagnosis of lung adenocarcinoma, comprising a detection reagent for a disintegrin and metalloproteinase domain-containing protein 32 (ADAM32) gene as a diagnostic marker for the lung adenocarcinoma.

2. The product according to claim 1, wherein the detection reagent is a reagent for determining an expression level of the ADAM32 gene in a biological sample.

3. The product according to claim 2, wherein the reagent for determining the expression level of the ADAM32 gene in the biological sample comprises a reagent for detecting a messenger RNA (mRNA) of the ADAM32 gene or a fragment thereof, a reagent for detecting a complementary DNA (cDNA) of the ADAM32 gene or a fragment thereof, and/or a reagent for detecting a protein encoded by the ADAM32 gene or a variant thereof.

4. The product according to claim 2, wherein the reagent for determining the expression level of the ADAM32 gene in the biological sample comprises primers for the ADAM32 gene and/or an ADAM32 antibody.

5. The product according to claim 4, wherein the primers for the ADAM32 gene are an upstream primer set forth in SEQ ID NO: 9 and a downstream primer set forth in SEQ ID NO: 10.

6. The product according to claim 1, wherein the product is a chip, a kit, or a test paper.

7. A drug for treating lung adenocarcinoma, comprising a reagent for inhibiting expression of an ADAM32 gene.

8. The drug according to claim 7, wherein the reagent for inhibiting the expression of the ADAM32 gene comprises an siRNA specifically targeting the ADAM32 gene, an antisense oligonucleotide specifically targeting the ADAM32 gene, a ribozyme specifically targeting the ADAM32 gene, and/or an inhibitory antibody for the ADAM32 gene.

9. The drug according to claim 8, wherein the inhibitory antibody for the ADAM32 gene is a monoclonal antibody, a polyclonal antibody, or a polyspecific antibody.

10. The drug according to claim 8, wherein the inhibitory antibody for the ADAM32 gene is the polyclonal antibody, and an antigen targeted by the polyclonal antibody is CHO-derived recombinant human ADAM32 Ser17-Thr476.

11. The drug according to claim 8, wherein the inhibitory antibody for the ADAM32 gene is an ADAM32 antibody of Thermo Fisher Scientific.

12. The drug according to claim 8, wherein nucleotide sequences for the siRNA specifically targeting the ADAM32 gene are SEQ ID NOS: 1 and 2, SEQ ID NOS: 3 and 4, or SEQ ID NOS: 5 and 6.

13. The drug according to claim 12, wherein the nucleotide sequences for the siRNA specifically targeting the ADAM32 gene are SEQ ID NOS: 3 and 4.

14. A drug screening system, comprising:
a detection module configured to detect an inhibitory effect of a drug on expression of an ADAM32 gene, and
an analysis module configured to output effect data of the drug for treating lung adenocarcinoma according to detection data output by the detection module.

15. A lung adenocarcinoma diagnosis system, comprising:
a detection device configured to detect an expression level of an ADAM32 gene in a biological sample of a subject, and
an analysis device configured to output a diagnosis result of lung adenocarcinoma of the subject according to data of the expression level of the ADAM32 gene output by the detection device.


SEQUENCE LISTING (10 sequences)

SEQ ID NO: 1 - RNA, 21 nt, synthetic construct
  source 1..21: mol_type = other RNA
  misc_feature 1..21: note = synthetic
gctctggactaagaggaattt

SEQ ID NO: 2 - RNA, 21 nt, synthetic construct
  source 1..21: mol_type = other RNA
  misc_feature 1..21: note = synthetic
attcctcttagtccagagctt

SEQ ID NO: 3 - RNA, 21 nt, synthetic construct
  source 1..21: mol_type = other RNA
  misc_feature 1..21: note = synthetic
gctgtcatcattggagttatt

SEQ ID NO: 4 - RNA, 21 nt, synthetic construct
  source 1..21: mol_type = other RNA
  misc_feature 1..21: note = synthetic
taactccaatgatgacagctt

SEQ ID NO: 5 - RNA, 21 nt, synthetic construct
  source 1..21: mol_type = other RNA
  misc_feature 1..21: note = synthetic
gccgaaagcacatcctgaatt

SEQ ID NO: 6 - RNA, 21 nt, synthetic construct
  source 1..21: mol_type = other RNA
  misc_feature 1..21: note = synthetic
ttcaggatgtgctttcggctt

SEQ ID NO: 7 - RNA, 21 nt, synthetic construct
  source 1..21: mol_type = other RNA
  misc_feature 1..21: note = synthetic
ttctccgaacgtgtcacgttt

SEQ ID NO: 8 - RNA, 21 nt, synthetic construct
  source 1..21: mol_type = other RNA
  misc_feature 1..21: note = synthetic
acgtgacacgttcggagaatt

SEQ ID NO: 9 - DNA, 20 bp, synthetic construct
  source 1..20: mol_type = other DNA
  misc_feature 1..20: note = synthetic
acccaaacacaaagcagtag

SEQ ID NO: 10 - DNA, 20 bp, synthetic construct
  source 1..20: mol_type = other DNA
  misc_feature 1..20: note = synthetic
gaccacggaaggaaagacag